(19) United States
(12) Patent Application Publication
(10) Pub. No.: US 2015/0038948 A1
(43) Pub. Date: Feb. 5, 2015

(54) Apparatus and use of a neurochemisrty regulator device insertable in the cranium for the treatment of cerebral cortical disorders

(71) Applicants: Nandor Ludvig, Astoria, NY (US); Geza Medveczky, Gortlandt Manor, NY (US); John G. Kral, Larchmont, NY (US); Sandor Toth, Mohegan Lake, NY (US)

(72) Inventors: Nandor Ludvig, Astoria, NY (US); Geza Medveczky, Gortlandt Manor, NY (US); John G. Kral, Larchmont, NY (US); Sandor Toth, Mohegan Lake, NY (US)

(73) Assignee: G-Tech Electronic Research & Development, LLC, Cortlandt Manor, NY (US)

(21) Appl. No.: 13/955,984

(22) Filed: Jul. 31, 2013

(51) Int. Cl. A61M 5/168

(52) U.S. Cl. CPC A61M 5/16804
USPC 604/891.1

(57) ABSTRACT

A subarachnoid pharmacodialysis apparatus insertable under the scalp, in and under the cranium, with a relatively short and simple neurosurgical procedure, to be kept there safely implanted for a year or longer for the purpose of regulating the neurochemistry of one or more diseased cerebral cortical areas and thus to achieve therapeutic effects via both localized delivery of medication and drainage of local neurotoxic molecules across the subdural meninges and compartments in a feedback-controlled fashion, with or without the additional capability of performing localized neurochemistry regulation in subcortical areas. This apparatus is also used for neurochemical profiling of the diseased brain area or areas by analyzing the removed endogenous molecules and adjusting the composition of the delivered medication based on the patient's specific, abnormal neurochemistry within the treated area or areas.

### FIELD OF THE INVENTION

This invention generally relates to treating neurological disorders with implanted devices, specifically those that direct fluids, such as drug solutions, cerebrospinal fluid (CSF) or secretions in and/or out of the brain and adjacent structures to achieve therapeutic effects.

### NOVELTY OF THE INVENTION

This invention is a single device, which is insertable in and under the cranial bone by a relatively short and simple neurosurgical procedure, to remain in place safely implanted for up to a year or longer for the purpose of regulating the neurochemistry of one or more diseased cerebral cortical areas using localized drug delivery and the drainage of extracellular neurotoxic molecules from the treated tissue via the subdural meninges and compartments to thus achieve therapeutic effects. This procedure is termed“subarachnoid pharmacodialysis”, since it involves both the delivery of pharmacological agents and the removal of harmful cortical molecules via dialysis through the subarachnoid space, across the pia mater acting as a dialysis membrane separating the subarachnoid space and the cerebral cortical tissue (Kral and Ludvig, 2012). The device is adaptable to include one or more deep brain cannulas or probes to extend the local neurochemistry-regulating function of the device to one or more deep brain areas, if this is necessary to achieve therapy in cerebral cortical disorders involving both neocortical and archicortical (e.g., hippocampal) or subcortical (e.g., striatal) pathologies. This concept has never before been used in intracranially implanted drug delivery pumps, polymers, microchips or other pharmacological devices insertable in and under the cranial bone since such devices have heretofore not had the capacity to remove extracellular neurotoxic molecules over long periods. Similarly intracranial shunts, intra- or extraventricular drains and other fluid removal devices have not had the capacity to modulate neurochemistry long-term by localized drug delivery to a pathological brain site. Thus, in contrast to implantable devices of the prior art, the present invention is suitable for both medication delivery and toxic molecule drainage from the brain within the course of treatment of a cerebral cortical disorder. This two-pronged strategy can create a favorable extracellular environment for the delivered drugs through clearing counteracting endogenous molecules and toxins, while, at the same time, amplifying the beneficial effects of pharmacological treatment of the ameliorated tissue. As a result, diseased cerebral cortical areas can be more effectively treated with the present invention than with implants of the prior art.

### BACKGROUND

The majority of neurological disorders have focal pathology localized to one or more areas of the Central Nervous System (CNS). Thus, about 30% of all treatment-resistant epilepsies (Callaghan et al., 2007), 30% of all ischemic strokes (Halkes et al., 2006), 85% % of malignant brain tumors (Larjavaara et al., 2007), and most traumatic brain injuries (TBI) are predominantly localized to the cerebral cortex. Progressive cognitive decline, the main clinical symptom of Alzheimer's disease, is also related to intra- and extracellular pathophysiological processes in the association cortex. Cerebral cortical disorders can involve both neocortical and deeper brain areas. For example, in temporal lobe epilepsy the temporal cortex and the archicortex (e.g., hippocampus) can both be responsible for seizure generation. At the time of the present disclosure, none of these devastating, often fatal, cerebral cortical disorders can be effectively treated with traditional pharmacological, neurosurgical, electrical stimulation, or other (e.g., behavioral, alternative, etc.) therapies.

The ultimate cause of the failure of traditional and current medical treatments for cerebral cortical disorders is that none of them are capable of correcting the neurochemical and molecular abnormalities that underlie the induction and/or maintenance of the disease at the site of the pathology. Systemic drug treatments cannot direct administered drugs specifically into the cortical site of pathology and achieve effectively high drug concentrations without causing serious systemic side-effects. Most traditional neurosurgical interventions can produce a wide variety of structural changes in and around the area of the cortical pathology, but lack the ability to correct or alter local neurochemistry. Electrical stimulation devices are also unable to selectively interfere with specific neurochemical and molecular mechanisms. The electrophysiological (e.g., action-potential-generating) and neurochemical (e.g., neurotransmitter-releasing) functions of cell transplants, engineered tissues and other biological implants are difficult to control, especially long-term, once surgically inserted in the brain, limiting the therapeutic efficacy of engineered neural tissue implants.

The present invention is a continuation of our prior U.S. Pat. No. 6,497,699 (Ludvig and Kovacs, 2002) and US Patent publication No. 20120053506. The apparatus and method disclosed here offers the ability to correct the neurochemical and molecular abnormalities that underlie the induction and/or maintenance of cerebral cortical disorders at the site of the pathology. As such, the present invention offers, among other therapeutic applications, the correction of excitatory-inhibitory imbalances in epileptic seizure foci, the promotion of neuroregenerative processes at the site of stroke or TBI, the prevention of the spread and neurotoxicity of malignant tumors, and neuromodulator replenishment coupled with neurotoxic molecule removal in Alzheimer's disease. The present invention, as built on the basic device architecture of our U.S. Pat. No. 6,497,699, also includes electrophysiological and/or neurochemical recording components, as well as bi-directional radiofrequency (RF) communication capability. This enables the neurochemistry regulating (subarachnoid pharmacodialysis) apparatus to obtain feedback data from the treated tissue for external transmission and analysis and/or online processing. In turn, this allows the present invention to (a) optimize the drug delivery—molecule removal parameters, and (b) adapt these parameters to changes in the treated area as the treatment evolves.

As an intracranial drug delivery device, the present invention relates to previous implantable devices and methods that aimed to provide pharmacological treatment for brain disorders by direct drug delivery into the neural tissue. Such prior devices and methods include U.S. Pat. No. 7,108,680, a closed-loop drug delivery system (Rohr et al., 2006); U.S. Pat. No. 7,241,283, an advanced intracranial catheter—pump apparatus (Putz, 2007); U.S. Pat. No. 7,931,899, an intracerebral infusion device for Alzheimer's disease (Shafer et al., 2011);U.S. Pat. No. 7,892,221, a drug release system where the drug molecules are dispersed from a degradable matrix in a controlled manner (Santini et al., 2011); and our previous U.S. Pat. No. 6,497,699 (Ludvig and Kovacs, 2002). The fundamental difference between this prior art, including our own U.S. Pat. No. 6,497,699, and the present invention is that whereas prior drug delivery devices were limited to aim therapeutic effects solely by pharmacological manipulations, such as drug delivery, the present apparatus and method can achieve therapeutic effects via both pharmacological manipulations and the removal of endogenous toxic molecules primarily from the extracellular environment of the diseased cortical area. As such, it works as a local, cerebral cortical neurochemistry regulator and pharmacodialysis device.

The importance of using both drug delivery into a diseased cerebral cortical site and the removal of potentially toxic molecules from this area might be appreciated by considering that the brain, while protected by the blood-brain barrier (BBB) and the cranium, lacks a lymphatic system. As a consequence, the elimination of potentially harmful endogenous molecules from the extracellular/interstitial space is less effective in the brain than from other organs. Although the extracellular/interstitial fluid in brain drains into the subarachnoid cerebrospinal fluid (CSF), thus providing a mechanism for the elimination of potentially harmful molecules in the epileptic seizure focus or in the area of an infarct, tumor or injury this clearance mechanism is compromised. Indeed, excess amounts of potentially neurotoxic amino acids and other molecules, such as nitric oxide (Ozden et al., 2011), harmful cytokines, such as IL-6, IL-1β, TNF-α (Vezzani et al., 2008), free radicals, such as superoxide (Oury et al., 1992), axonal growth inhibitory factors, such as Nogo-A and chondroitin sulfate proteoglycans (Harris et al, 2009; Permet and Schwab, 2012) are present in the extracellular environment of diseased cerebral cortical areas. In Alzheimer's disease, soluble extracellular amyloid-βA oligomers are known to cause neurotoxic effects (Selkoe, 2008; Sakano and Zako, 2010). The present invention provides an apparatus and method for the periodic removal of these molecules, normalizing the extracellular neurochemical milieu and creating favorable conditions for the effects of the delivered drugs. In addition, the present invention can also be used as non-externalized or closed drainage for cortical debris and inflammatory cells and molecules (e.g., lymphocytes, cytokines, etc.), for weeks or months, to complete the neurosurgical treatment of hemorrhagic strokes and traumatic hemorrhages. Since these neurosurgical treatments require craniotomy, complementing such craniotomies with the implantation of the present device would only be a minimal addition to the neurosurgical procedure. The fact that this drainage is non-externalized (albeit accessible percutaneously) can reduce or eliminate the risk of infection. Furthermore, the use of a pump mechanism for drainage or flushing makes it possible to facilitate the removal of potentially harmful cells and molecules from the area of hemorrhage, promoting local cellular recovery. Importantly, the dual process of medication delivery and neurotoxic molecule drainage takes place in the area of cerebral cortical pathology (e.g., cortical infarct, degenerating association cortex, neocortical seizure focus, etc.) providing maximum exposure of the targeted cells to medication and maximum efficacy for clearing toxic molecules from the local extracellular space.

The present invention differs from our prior US Patent publication No. 20120053506 that in the present device the control unit is (a) insertable in the cranial bone overlaying the affected cortical area and (b) it forms a single continuous apparatus with the subdural fluid exchange unit. As a consequence, the neurosurgical implantation requires only a single craniotomy without (1) the need of any separate, remote implantation site for a control unit and (2) without the need of connecting the subdural and control units with subcutaneously tunneled tubing. These improvements greatly simplify the neurosurgical procedure, allowing the neurosurgeon to avoid the risks of a long and complicated operation. The mentioned improvements also eliminate the risk of damage in the tubing system, as the intracranial connecting tubing between the control and subdural units is subjected to no twisting, pulling or other agitations by body movements: mechanical effects that can bedevil the use of subcutaneously tunneled fluid tubing. The present invention further differs from our prior US Patent publication No. 20120053506 in that the present subdural fluid-exchange unit can also function as a scaffold for pre-surgically seeded autologous cells, such as fibroblasts, mesenchymal stem cells or other autologous cells (harvested from the patient). This should prevent excessive inflammatory tissue reaction after the subdural unit is implanted.

As the device performs both drug delivery into the diseased cortex and the removal of local endogenous molecules from the treated cortical site, this also allows the neurochemical profiling of the diseased area and, in response, tailoring the composition of the delivered drug solution to the patient's specific neurochemical abnormality. This ability can further increase the therapeutic efficacy of the neurochemistry regulator device.

### SUMMARY OF THE INVENTION

The present invention relates to the treatment of cerebral cortical disorders by a device that regulates local neurochemistry primarily in and around a diseased cerebral cortical area (e.g., seizure focus, infarct, etc.) with the dual mechanism of dispensing drug solutions into this area while also removing potentially harmful endogenous molecules from the site of pathology. This is accomplished by the use of a dual minipump capable of both directing fluids into the cortical tissue and removing potentially toxic molecules from the cortical extracellular environment, performing pharmacodialysis. Accordingly, the control unit of the device comprises the mentioned dual minipump, a battery, and a microcontroller integrated with a radiofrequency (RF) communication module. The entire control unit is insertable in the cranial bone, under the scalp and overlying the cortical site of pathology, connected with a short, flexible conduit running under the cranial bone and dura mater to the subdural unit. It is this subdural unit through which drug molecules diffuse into the cortex and neurotoxic molecules diffuse from the cortical extracellular space into the CSF-filled subarachnoid space. As in our prior Patent publication No. 20120053506, the subdural unit is equipped with electrophysiological recording electrodes to provide feedback on the cerebral cortical effects of the device, allowing the adjustment and fine-tuning of the parameters of drug delivery and endogenous molecule removal. The subdural unit is suitable to function as a scaffold with its surface covered with a thin layer of seeded autologous tissue to prevent inflammatory tissue reactions. The control unit is covered with a shell that hermetically closes the gap between the device and the cranial bone. Two subcutaneous fluid ports, protruding from the shell, serve to refill the drug reservoir component of the minipump and empty the molecule collector component of the minipump, through the scalp, when needed. The device is adaptable to include one or more deep brain cannulas or probes to extend the local neurochemistry-regulating function of the device to one or more deep brain areas, if this is necessary to achieve therapy in cerebral cortical disorders involving both neocortical and archicortical (e.g., hippocampal) or subcortical (e.g., striatal) pathologies.

### BRIEF DESCRIPTION OF THE DRAWINGS

FIG. 1 illustrates the scientific principle of the invention applied to the treatment of cerebral cortical disorders.

FIG. 2 presents a schematic diagram of the invention and its spatial relationship to the cerebral cortex.

FIG. 3 shows the preferred design of the invention's control unit.

FIG. 4 depicts the subdural unit, in this version equipped with an electrode-cannula.

FIG. 5 presents a flow-chart from collection of fluid for neurochemical profiling of the affected cortical area to delivery of tailored pharmacotherapy into the area in question.

### DETAILED DESCRIPTION

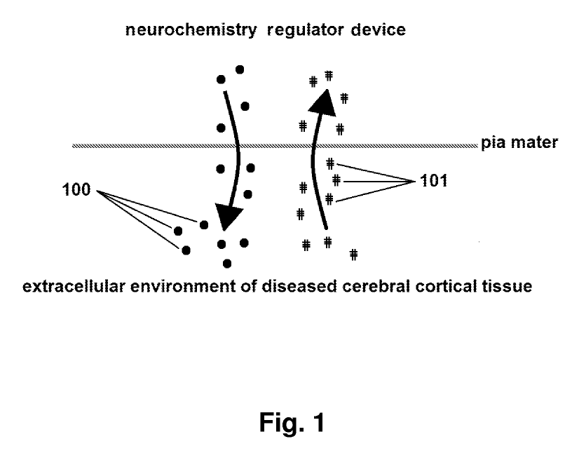

The following description and related appended drawings, wherein like elements are provided with the same reference numerals, further explains the present invention. The present invention fully utilizes the scientific principle of bi-directional molecule diffusion across the cerebral cortical pia mater according to Fick's laws of diffusion, allowing both drainage of potentially toxic molecules from the cortical extracellular/interstitial space into the overlying subarachnoid compartment and penetration of drug molecules from the subarachnoid space into the underlying cortical extracellular/interstitial space, as also demonstrated experimentally in vivo (Wang et al., 1983; Ludvig et al., 2012a). FIG. 1 illustrates this principle and its relation to the present invention by showing the opposite directions of movement of drug molecules 100 from the neurochemistry regulator device into the diseased cortex through the pia mater and exit of the potentially toxic cortical extracellular molecules 101 from the diseased cortex across the pia mater to the same neurochemistry regulator device.

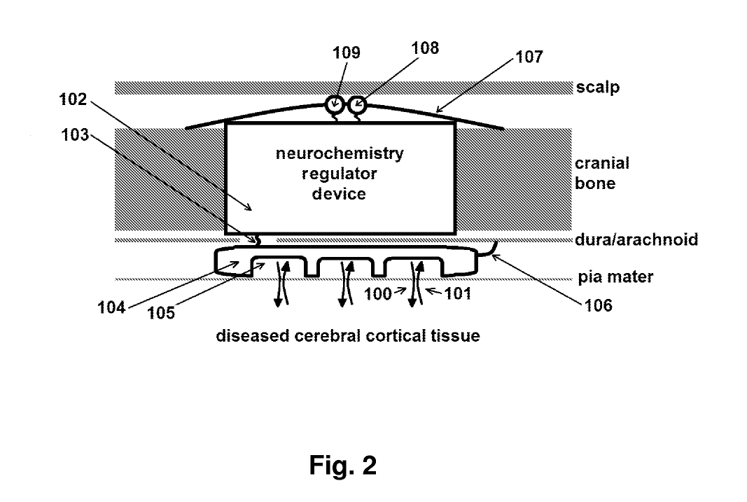

The system of the present invention, designed to utilize the principles of (0016) for therapeutic purposes, is demonstrated in FIG. 2. The control unit 102 of the neurochemistry regulator device, comprising the dual minipump mentioned in 0010 and a battery-powered microcontroller equipped with an RF communication module, is embedded in flexible plastic or other material adaptable to the shape of the craniotomy over the diseased cerebral cortical tissue to be treated. Adaptation of this flexible embedding material to the shape of the craniotomy can be guided by CT, MRI and other neuroimaging data obtained from the patients before surgical device implantation, allowing sufficient time for the manufacturer to make this patient-tailored adaptation in the shape of control unit 102. One end of the control unit 102 is connected to a short, flexible, impermeable conduit 103 to the subdural unit 104. Subdural unit 104 mediates the drug delivery—subarachnoid fluid removal procedure and electrophysiological and/or neurochemical recordings described in our prior US patent publication No. 20120053506, as well as the opposite movement of therapeutic drug molecules 100 and endogenous toxic molecules 101 across the pia mater, as shown in FIG. 1. The conduit includes tubing and recording wires running between the control unit 102 and the subdural unit 104. The subdural unit 104 is placed on the pia mater covering the diseased cerebral cortical tissue after having retracted the dura/arachnoid to expose the pial/cortical surface. Once the subdural unit 104 is placed, the dura/arachnoid is re-positioned to its original site and closed with sutures around conduit 103. This can be followed by anchoring the subdural unit 104 to the already overlaying dura mater with one or more hooks 106 made of the same biocompatible medical grade material as the subdural unit 104. The hook 106 can be anchored by locating it under the dura, making a small incision on the overlaying dura, and suturing the hook to the dura. The hook 106 under the dura can be palpated or localized using hooks incorporating a temporary light source such as a fiber optic wire removable after suturing the hook to the dura.

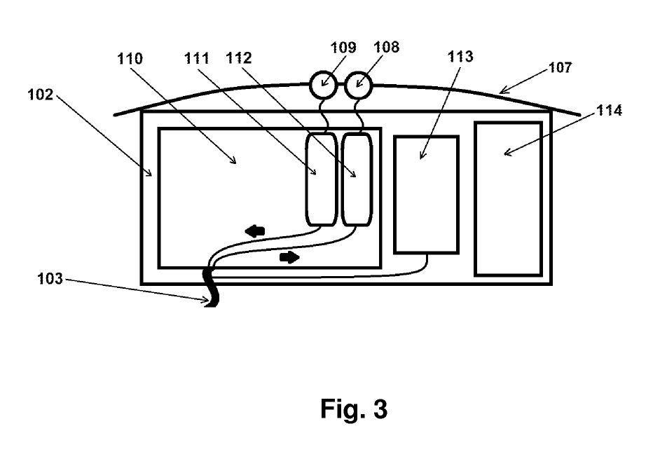

The control unit 102 of the invention is further detailed in FIG. 3. As mentioned in (006) and (0010), the present invention is a continuation of the applicants' U.S. Pat. No. 6,497,699 and US Patent publication No. 20120053506. Therefore, the design of the control unit 102 is consistent with that of these prior inventions. Accordingly, the control unit comprises a dual minipump 110. This minipump incorporates a drug delivery pump and reservoir 111, which directs the flow of the drug solution (upper thick arrow) toward the brain via tubing enclosed in the conduit 103. The reservoir 111 is percutaneously refillable through port 109. The minipump also incorporates a second pump component 112 that drains fluid from the subarachnoid space (lower thick arrow), and via this process, from the extracellular/interstitial space of the underlying cortical tissue. The fluid is collected in the reservoir of this second pump component 112 and can be permanently eliminated by percutaneous aspiration from the device through port 108. The control unit further comprises a microcontroller 113 integrating a RF communication module, and a battery 114. What distinguishes this control unit design from that of U.S. Pat. No. 6,497,699 and US Patent application 20120053506 is that in the present invention the control unit 102 is attached to a protective cover 107, made of titanium or other durable biocompatible material, which both houses the subcutaneous fluid ports (108 and 109) and allows the neurosurgeon to secure the control unit to the cranial bone and hermetically close and seal the craniotomy with screws and/or biocompatible glue applied between the cover 107 and the bone. In this way, the control unit 102 of the neurochemistry regulator device becomes an integral part of the cranium over the cortical site of treatment, performing therapeutic functions while also serving as a cranial bone replacement to protect the treated cerebral cortical area. The exact spatial relationship between the control unit 102 and the dual minipump comprising the drug delivery pump/reservoir 111, the drug refilling port 109, the fluid drainage pump/reservoir 112 and the fluid aspiration port 108 can be different from the preferred arrangement shown in FIG. 3. For example, the reservoirs and their associated ports can be placed outside of the rest of the control unit 102 to occupy an extended portion of the adjacent cranium and thus hold increased volumes of fluids. In this case components 108, 109, 111 and 112 are encased in their own dedicated shell and connected to control unit 102, still forming a single device, with insulated tubing running within the cranium.

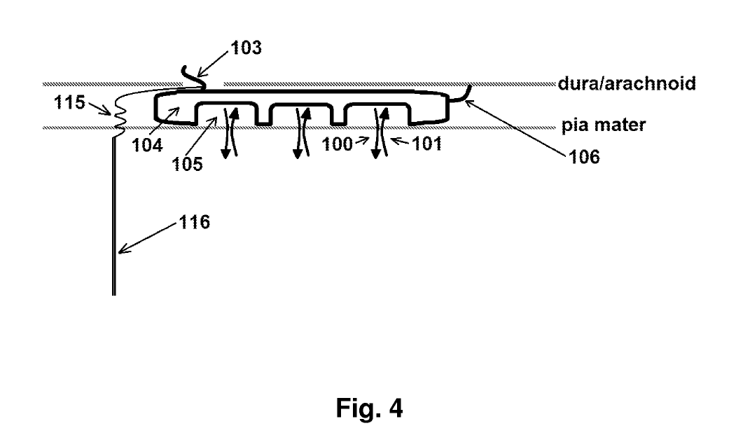

One version of the subdural unit 104 is depicted in FIG. 4. Consistent with the prior invention of US Patent publication No. 20120053506, the subdural unit 104 is shaped as a less than 1.5 mm thick strip or grid, depending on the size and geometry of the treated cortical area, and it comprises sealed fluid exchange ports 105 to mediate the movement of drugs 100 and endogenous molecules 101 through the subarachnoid space to and from the treated cortex, respectively. Since these molecules are dissolved and moved in fluids, the subdural unit 104 functions as a fluid-exchanging device. The subdural unit 104 can be equipped with recording electrodes and/or neurochemical sensors. These electrodes and sensors can be either placed at a distance from the sealing rim of the fluid ports, separated from these ports, as disclosed in US Patent publication No. 20120053506 or can be integrated parts of the sealing rim of the fluid ports. Wiring from these electrodes and/or sensors run within the impermeable conduit 103 connected to the control unit 102. The subdural unit 104 can be sutured to the overlaying dura mater with one or more hooks 106, as described above (0017). What distinguishes the present subdural unit from similar, previously disclosed units of subdural pharmacotherapy devices (Ludvig et al., 20012b,c; US patent publication No. 20120053506) is that the present invention provides the option of combining the subdural unit with a deep-brain electrode-cannula apparatus, similar to what was described in the applicants' U.S. Pat. No. 6,497,699. Thus, a flexible cable 115 that includes electrode wiring and fluid tubing can be led out of the conduit 103 and connected to the electrode—cannula apparatus 116. In turn, this apparatus 116 can be stereotaxically introduced into deep brain areas, such as the hippocampus, to pharmacologically prevent seizure genesis in temporal lobe epilepsy, improve memory-consolidating hippocampal functions in Alzheimer's disease, or for the pharmacological treatment of subcortical areas involved in the pathophysiology of Parkinson's disease and multiple sclerosis. One or more electrode—cannulas 116 can be added to the subdural unit 104. The cannula component can be a single cannula or the intraparenchymal microprobe suitable for both drug delivery and extracellular fluid removal, as disclosed in prior US Patent publication No. 20110071425 by Ludvig et al. in 2011.

While the subdural unit 104 can exclusively be constructed from biocompatible materials, such as medical grade silicone and platinum, the resulting structure can also be used as a scaffold to accommodate one or more layers of autologous cells. These cells can derive from the patient to be implanted by the neurochemistry regulating device, seeded into the scaffold ex-vivo, and allowed to grow and proliferate to form one or more layers on the exterior surface before this process is terminated, readying the device for implantation. During the ex-vivo treatment of unit 104, the lumen of its fluid-port 105 and that of conduit 103, as well as the tubing in between these parts, are plugged with multiple replaceable plugs to prevent unwanted cell growth into the channels of fluid movement. The plugs are removed prior to implantation.

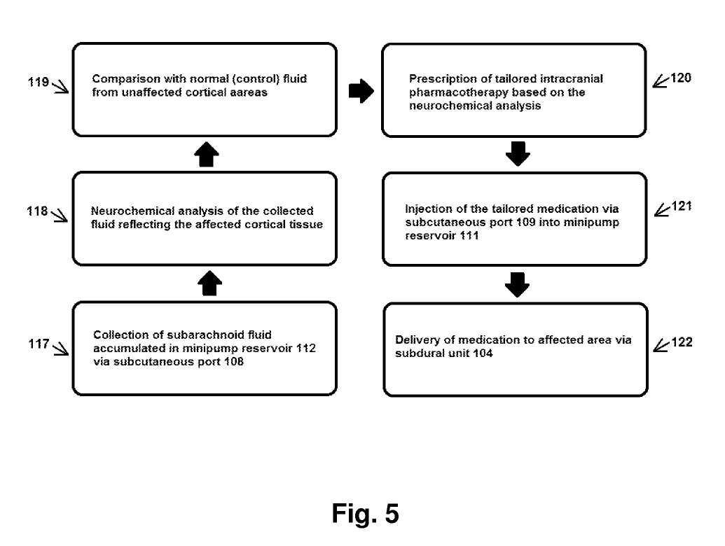

The ability of this invention to remove subarachnoid fluid directly from the site of cerebral cortical pathology offers 3 distinct advantages. First, as described in prior US Patent publication No. 20120053506, as well as in subsequent published materials (Ludvig et al., 2012b,c), this procedure prevents the drug delivery system from clogging by the virtue of washing out inflammatory proteins and cells before they can aggregate. Second, as described in the present application (007), this procedure also removes potentially neurotoxic and growth inhibitory endogenous extracellular molecules (e.g., excess glutamate, IL-6, Nogo-A, etc.) drained into the local subarachnoid fluid. Third, by analyzing the neurochemical composition of the extracellular/subarachnoid fluid accumulated in the second minipump reservoir 112 and permanently eliminated through subcutaneous port 108, information can be obtained on the neurochemical abnormality specific to the diseased cerebral cortical area. In turn, this neurochemical profiling, ideally performed with a range of biochemical assays for measuring small molecules, peptides and proteins e.g. with HPLC, ELISA, 2-D gel electrophoresis, mass spectrometry, and/or other techniques, can help to rationally devise the composition of the therapeutic solution for a prescription to be delivered via the drug delivery pump component 111. This fine-tuning of the localized drug treatment should increase therapeutic efficacy. Flow-chart in FIG. 5 indicates the steps 117, 118, 119, 120, 121, 122 to adjust drug delivery based on data generated by the neurochemical analysis of the collected affected subarachnoid fluid.

This invention is suitable for delivering drugs either through the subdural unit 104 alone or via both this unit and one or more attached electrode—cannulas 116. The temporal order of drug delivery can either be predetermined, producing automatic drug administration continuously, intermittently or in a periodic fashion, or on-demand, in response to electrophysiological signals detected by the recording electrodes or to neurochemical signals detected by the neurochemical sensors, as described in the US Patent publication No. 20120053506, on which this invention is built. Mixed automatic and on-demand drug delivery modes can also be performed.

The delivered drug solution can contain a single drug or a mixture (cocktail) of various compounds. For example, the delivered solution for focal seizure prevention can contain only muscimol or another antiepileptic agent, whereas the therapeutic solution for Alzheimer's disease can contain a mixture of neurostimulators (e.g., acetylcholine, etc.), neuromodulators (e.g., norepinephrine, etc.), neuroregenerative compounds (e.g., Nerve Growth Factor, etc.), and/or neurogenesis promoting agents (e.g., metformin, etc.).

Besides medication delivery, this invention is suitable for removing extracellular fluid and its dissolved endogenous molecules, including toxic ones, through the subdural unit 104 alone or via both this unit and one or more attached electrode—cannulas 116 if the cannula is structured similarly to the microprobe referred to in (0019). In this way, diseased cerebral cortical areas receiving pathophysiological extracortical inputs can also be treated. The temporal order of subarachnoid fluid removal can also be either predetermined, producing automatic fluid removals continuously, intermittently, periodically, or on-demand, in response to electrophysiological signals detected by the recording electrodes or to neurochemical signals detected by the neurochemical sensors. Mixed automatic and on-demand fluid removals can be performed, as well. Drug delivery and fluid removal are performed in an alternating fashion, with the interval separating the delivery and removal periods flexibly adjustable from seconds to days. When a drug solution is delivered through the subdural unit 104, the used solute is dissolved in artificial CSF. Thus, it is this solution that fills the subarachnoid space over the treated cortical area. By changing the neurochemical composition of the artificial CSF, it is possible to control the chemical concentration gradient that drives the various cerebral cortical endogenous molecules through the pia mater and subarachnoid space into the fluid-exchange ports 105. In turn, this allows the differential removal of specific molecules or molecular sets from the diseased cortical area and the consequent flow of harmful endogenous molecules into the invention's drain pump 112. For example, if the treatment of the diseased area does not require the removal of excess extracellular glutamate but does require the removal of pro-inflammatory cytokines, then the artificial CSF solvent is adjusted to contain no pro-inflammatory cytokines but the same concentration of glutamate as what is present in the cortical extracellular space. As a consequence, the chemical concentration gradient for glutamate is cancelled, leading to no flux from the cortex through the pia mater, whereas the chemical concentration gradient for the pro-inflammatory cytokines is maintained, leading to flux for these proteins from the cortex through the pia mater.

Built on our prior US Patent publication No. 20120053506, the present invention can be equipped with electrophysiological and/or neurochemical recording capability, as mentioned in (0010). This allows the device to deliver the therapeutic compounds in response to abnormal EEG signals and/or to adjust the delivery parameters according to characteristics of the recorded signals. These signals can be either analyzed online by the microcontroller or transmitted via the RF module for external analysis. Based on either of these analyses, the microcontroller can adjust and optimize the delivery and drainage parameters of the dual minipump. For example, focal epilepsy treatment can be performed by initially instructing the dual minipump to deliver and drain fluids periodically at pre-fixed intervals, but once the seizures stop, the dual minipump is switched to deliver the antiepileptic medication only when interictal spikes appear, as proposed (Ludvig, 2002). This is a mixed, automatic/on-demand drug delivery mode, based on the online monitoring of interictal EEG spikes: a strategy different from both currently used EEG seizure recognition/detection and seizure prediction/anticipation methods which rely on ictal or pre-ictal signal monitoring and not on interictal signal monitoring. Another application of EEG monitoring with the neurochemistry regulator device is the occasional (e.g., weekly) transmission of brief, (e.g. 10-min) intracranial, cortical EEG segments with the RF module for external analysis. This analysis can fine-tune the parameters of the delivered drug-cocktail so that the treatment can increase the high-frequency (>12-20 Hz) components of the EEG activity during daytime, when this is needed to maintain effective cognitive functions. At the same time, the analysis can recognize abnormal EEG signals, such as EEG spikes or flattening, allowing the adjustment of drug dosing to normalize local electrophysiological processes before they lead to clinically significant side-effects.

What is claimed is:

1. A neurochemistry regulating pharmacodialysis device comprising (a) a control unit insertable in/under the cranial bone, and (b) a fluid-exchanging subdural unit implantable over a diseased cerebral cortical area to restore the area's physiological functions by delivering medication via the subarachnoid space into the affected cortical cells' environment and/or removing potentially toxic, extracellular endogenous molecules from the same diseased area via the same subarachnoid route.

2. The apparatus of claim 1, where the subdural unit contains multiple, sealed fluid-exchanging ports and tubes to allow the movement of medications and extracellular endogenous molecules in and out of the cortical tissue exclusively via the subarachnoid space and pia mater overlying the diseased cortical area.

3. The apparatus of claim 1, where the subdural unit contains electrophysiological recording electrodes and/or neurochemical sensors, either integrated with the fluid ports or separated from them, to provide feedback on the effects of drug delivery and extracellular molecule removal in the treated cortical area.

4. The apparatus of claim 1, where the subdural unit is covered with a non-proliferating layer of ex-vivo grown autologous cells to prevent inflammatory tissue reactions without interfering with fluid movement in the fluid-exchanging ports and tubes.

5. The apparatus of claim 1, where the subdural unit is connected to the control unit with a short, flexible and impermeable conduit to form a single continuous device for neurosurgical implantation.

6. The apparatus of claim 1, where the connecting conduit includes fluid tubing and electrode- or sensor-wiring to and from the subdural unit.

7. The apparatus of claim 1, where the connecting conduit stays below the cranium, eliminating the need of subcutaneous tunneling between the control unit and the subdural unit.

8. The apparatus of claim 1, where the subdural unit is secured to the overlying dura mater with one or more flexible hooks sutured to the dura.

9. The apparatus of claim 1, where more than one subdural units are connected to the control unit via a branching conduit to treat more than one diseased cerebral cortical site.

10. The apparatus of claim 1, where the control unit comprises a dual minipump, a microcontroller, an RF communication module, each powered by a battery and embedded, separately but connected with wires, in a flexible material to allow the shaping of the curvature of the control unit so that it can be inserted in the cranium.

11. The apparatus of claim 1, where the dual minipump comprises a drug delivery component connected to a drug-refilling subcutaneous port and a subarachnoid-fluid-collecting component connected to a fluid-disposal subcutaneous port.

12. The apparatus of claim 1, where the control unit is attached to a protective cover shaped to follow the curvature of the control unit.

13. The apparatus of claim 1, where the protective cover of the control unit is structured to be attachable to the cranium hermetically and without applying pressure on the subdural unit.

14. The apparatus of claim 1, where the protective cover integrates the drug-refilling port and the subarachnoid fluid disposal port of the dual minipump so that these ports can be accessed through the overlying scalp.

15. The apparatus of claim 1, where all or some parts of the dual minipump are placed outside of the main body of the control unit, as extensions embedded in adjacent portions of the cranium, and connected to the control unit with insulated tubing, still forming a single device but one that can hold larger volumes of fluids than a compact apparatus without extensions.

16. The apparatus of claim 1, where the control unit is connected via a branching conduit to one or more subdural units and one or more deep-brain electrode-cannulas to perform treatment in multiple cortical and extracortical sites.

17. The apparatus of claim 1, where the cannula component of the deep-brain electrode-cannulas is a microprobe allowing both drug delivery and extracellular fluid removal in the implanted deep brain area.

18. A method of using the apparatus of claim 1 for months or years to allow the completion of the treatment of the cerebral cortical disorder with the neurochemistry regulator device.

19. The method of claim 18, wherein the apparatus is periodically refilled with medication through the subcutaneous refilling port, followed or preceded by disposal of the collected subarachnoid fluid through the corresponding subcutaneous port.

20. The method of claim 18, wherein the signals from the electrophysiological recording electrodes and/or neurochemical sensors are transmitted by the RF module of the apparatus for off-line or on-line examination to determine the safety and efficacy of the treatment with the apparatus.

21. The method of claim 18, wherein the electrophysiological and/or neurochemical signals transmitted by the RF module of the apparatus for off-line or on-line examination are used to flexibly change the composition of medication and/or change the parameters of medication delivery with the apparatus, if necessary, to keep the treatment schedule optimized.

22. The method of claim 18, applied specifically for epilepsy treatment, wherein the microcontroller selectively detects interictal EEG spikes to allow the delivery of the antiepileptic drug solution in response to the sustained occurrence of such interictal spikes over a pre-determined time-window and to repeat the antiepileptic drug delivery at pre-determined intervals until the interictal EEG spikes cease to occur.

23. The method of claim 18, applied specifically for Alzheimer's disease treatment, wherein the EEG activity transmitted by the RF module is analyzed off-line to separate the various frequency components of the EEG waves, determine their power, and adjust the parameters of drug delivery so that this treatment can increase or decrease the power of high and low frequencies over the course of each day in a manner that is optimal for effective cognitive performance.

24. The method of claim 18, wherein the control unit directs fluid movement in the subdural unit to alternately perform drug delivery and the removal of potentially toxic endogenous molecules in the diseased cortical area.

25. The method of claim 18, wherein the therapeutic solution delivered at any time by the device contains a single solute of a synthesized or naturally occurring compound.

26. The method of claim 18, wherein the therapeutic solution delivered at any time by the device contains a mixture of synthesized and/or naturally occurring compounds to exploit their synergistic actions and increase therapeutic efficacy.

27. The method of claim 18, wherein the control unit simultaneously directs drug delivery through more than one subdural units to perform drug treatment in multiple cerebral cortical sites.

28. The method of claim 18, wherein the control unit simultaneously directs drug delivery through one or more subdural units and one or more deep-brain electrode-cannulas to perform drug treatment in multiple cerebral cortical and extracortical sites.

29. The method of claim 18, wherein the control unit simultaneously directs fluid movement out of the subarachnoid space through more than one subdural units to remove potentially toxic endogenous molecules from multiple cortical sites.

30. The method of claim 18, wherein the control unit simultaneously directs fluid movement out of the subarachnoid space through one or more subdural units and one or more deep-brain electrode-cannulas to remove potentially toxic endogenous molecules from both cerebral cortical and extracortical sites.

31. The method of claim 18, wherein the subarachnoid fluid samples eliminated from the fluid disposal subcutaneous port are processed for off-line neurochemical analyses for neurochemical profiling of the diseased cerebral cortical area.

32. The method of claim 18, wherein the results of the neurochemical profiling are used for tailoring the composition of the applied medication to the patient-specific, abnormal neurochemistry of the treated area and thus fine-tuning the drug treatment.

33. The method of claim 18, wherein the depth of drug penetration into the cortical or extracortical parenchyma is increased by increasing the hydrostatic pressure applied with the drug delivery component of the minipump.

34. The method of claim 18, wherein the temporal order of drug delivery is either predetermined, producing automatic drug administration continuously, intermittently or in a periodic fashion, or on-demand, in response to electrophysiological signals detected by the recording electrodes or to neurochemical signals detected by the neurochemical sensors.

35. The method of claim 18, where the control unit is exclusively used as a non-externalized drainage system for the removal of harmful molecules and inflammatory products from brain tissue.

36. The method of claim 18, wherein the neurochemical composition of the artificial CSF solvent, which is used for the delivered drugs and fills the treated cortical area's subarachnoid space, is adjusted to increase or decrease the chemical concentration gradient for specific cortical molecules or molecular sets and thereby allow the differential removal of potentially harmful endogenous molecules from the diseased cortical extracellular space.

37. The method of 18, wherein it is used for the treatment of stroke, traumatic brain injury, brain tumor, epilepsy, Alzheimer's disease, and other neurological and psychiatric disorders, including drug addiction, for which existing alternative therapies are less effective than therapy with the apparatus of claim 1.
